(19) United States
(12) Patent Application Publication
(10) Pub. No.: US 2018/0001015 A1
(43) Pub. Date: Jan. 4, 2018

(54) AUTO-INJECTOR CASE

(71) Applicant: ULRIKE H M ZIEGNER, Palos Verdes Estates, CA (US)

(72) Inventor: ULRIKE H M ZIEGNER, Palos Verdes Estates, CA (US)

(21) Appl. No.: 15/201,612

(22) Filed: Jul. 4, 2016

(51) Int. Cl. A61M 5/00; A45F 5/00; B65D 81/38; B65D 83/00; B65D 83/02

(52) U.S. Cl. CPC A61M 5/002; B65D 83/0038; B65D 83/02; B65D 81/3816; A45F 5/00; A45F 2005/006; A61M 2205/3368; A61M 2209/088

(57) ABSTRACT

An insulated, epinephrine auto-injector case that also includes a rapid-opening cap, two spring-loaded chambers, each with a rapid-opening cap to facilitate immediate and easy availability of the auto-injector for grasping by the user, means for recording the expiration date of an auto-injector contained within the case, such as a re-writeable surface positioned on the case, means for attaching the case to a user or to a user's clothing, such as a stitched holster, a belt loop, a belt clip or an adjustable lanyard.

### CROSS REFERENCE TO RELATED APPLICATION

This application is a continuation-in-part of U.S. application Ser. No. 13/920,019, filed Jun. 17, 2013, now U.S. Pat. No. 9,381,294, and claims the benefit of priority of U.S. application Ser. No. 13/920,019, U.S. provisional patent application 61/672,460, filed Jul. 17, 2012, U.S. provisional application 61/660,760, filed Jun. 17, 2012, and each of which above-identified applications is incorporated by reference herein.

### FIELD OF INVENTION

The invention relates generally to cases for medication delivery devices such as auto-injectors used for injection of epinephrine.

### BACKGROUND

Cases for medication delivery devices such as auto-injectors used for injection of epinephrine are well-known. However, no known conventional epinephrine auto-injector case provides an insulated chamber that protects the epinephrine from degradation by extreme or prolonged temperature variance from ambient temperature. Also, no known conventional epinephrine auto-injector case provides a spring-loaded, internal container or chamber that forces the contained auto-ejector immediately upon opening an internal, rapid-release cap. Also, no known epinephrine auto-injector case provides for sensing and recording the temperature inside of the case to provide information on degradation or loss of effectiveness of the encased epinephrine due to prolonged storage in high temperature environments.

### SUMMARY

The auto-injector cases according to the present invention overcome the drawbacks of known auto-injector cases by providing a spring-loaded and/or insulated chambers for two injectors to facilitate rapid availability of each injector for grasping by the user, and a pop-open cap to provide secure containment of each injector within an insulated, protective case. Preferably the cap is of a design that provides for rapid access to the injector, and preferably the case is sized and configured to house two injectors, although it may also be adapted for a single injector. In another preferred embodiment a temperature measuring device or strip of material is included on the interior of the case to monitor and record the temperature inside of the case so that the user can be informed if the injector has been heated to a degree that renders its epinephrine of reduced effectiveness or ineffective.

Preferred embodiments of the present invention also may include means for recording the expiration date(s) of the auto-injector(s) contained within the case. The means for recording can and may be any conventional structure and processes, such as a re-writeable plate or surface upon which an expiration date may be written, numbered dials or wheels positioned in a holder and adapted to be set to an expiration date. Preferred embodiment cases may also include means to be attached to a user or to a user's clothing, such as a case having a belt loop placed directly on the back side of the case, a stitched holster having a belt loop and an adjustable lanyard that may be attached to the case and looped around the neck of a user.

Several embodiments, features, aspects, and advantages of the invention will become better understood with regard to the following description, appended claims and accompanying drawings.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a front perspective view of a preferred embodiment case for a conventional EpiPen® brand epinephrine auto-injector;

FIG. 2 is a perspective view of the FIG. 1 embodiment with the case top cap open and showing a closed cap for each of two contained auto-injectors;

FIG. 3 is a cross-sectional view of the FIG. 1 embodiment taken through line 3-3 of FIG. 2;

FIG. 4 is a rear perspective view of the FIG. 1 case, showing a belt clip;

FIG. 5 is a front perspective view of a conventional case and an EpiPen® brand epinephrine auto-injector showing how the injector is inserted and withdrawn from the case;

FIG. 6A is a perspective view of an alternate embodiment epinephrine auto-injector case having a re-writable strip adapted to write and re-write an expiration date;

FIG. 6B is a side view of the FIG. 6A embodiment;

FIG. 6C is a top view of the FIG. 6A embodiment;

FIG. 7A is a front perspective view of an alternate embodiment epinephrine auto-injector case having rotatable dials adapted to set and show an expiration date;

FIG. 7B is front view of the FIG. 7A embodiment;

FIG. 7C is a top view of the FIG. 7B embodiment;

FIG. 8A is a front perspective view of an alternate embodiment epinephrine auto-injector case with a stitched belt holster;

FIG. 8B is a front view of the FIG. 8A embodiment;

FIG. 8C is a side view of the FIG. 8A embodiment;

FIG. 9A is a rear perspective view of an alternate embodiment epinephrine auto-injector case with a removable/replaceable belt clip;

FIG. 9B is a side view of the FIG. 9A embodiment;

FIG. 9C is a top view of the FIG. 9A embodiment;

FIG. 10A is a front view of an alternate embodiment epinephrine auto-injector case with an adjustable lanyard shown hung around the neck of a user;

FIG. 10B is a side view of the FIG. 10A embodiment; and,

FIG. 10C is a back side perspective view of the FIG. 10A embodiment.

FIG. 10D is a perspective view of the lanyard fastener of the FIG. 10A embodiment.

Reference symbols or names are used in the Figures to indicate certain components, aspects or features shown therein. Reference symbols common to more than one Figure indicate like components, aspects or features shown therein.

### DETAILED DESCRIPTION

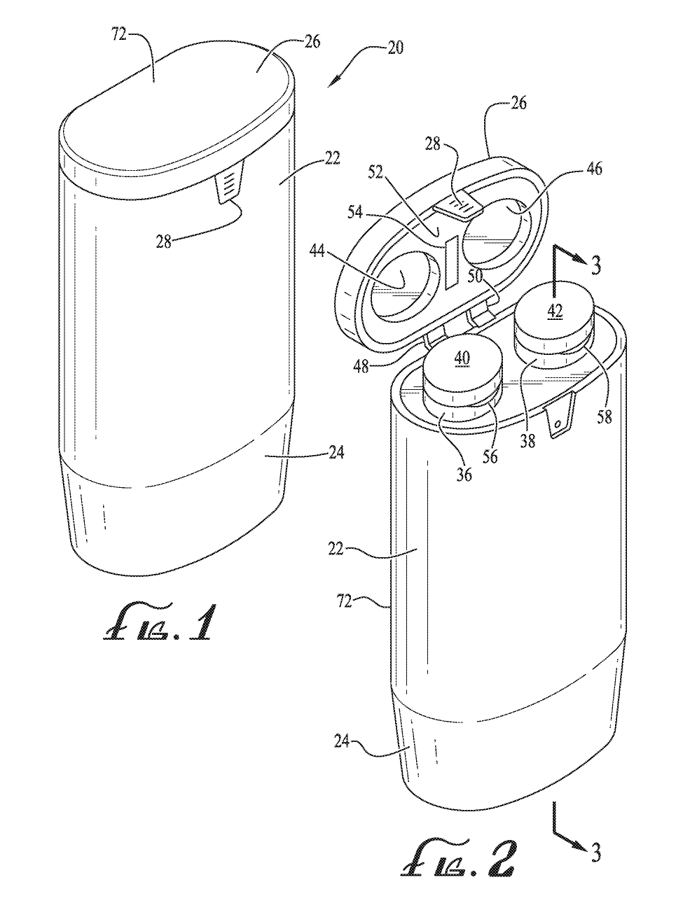

With reference to FIGS. 1-4 preferred embodiments of cases for auto-injectors are described. Case 20 preferably is sized and adapted to hold two EpiPen® brand epinephrine auto-injectors in a side-by-side relationship. The case 20 has a main body 22, a tapered bottom part 24, and a rapid-open top cap 26 with a tab 28.

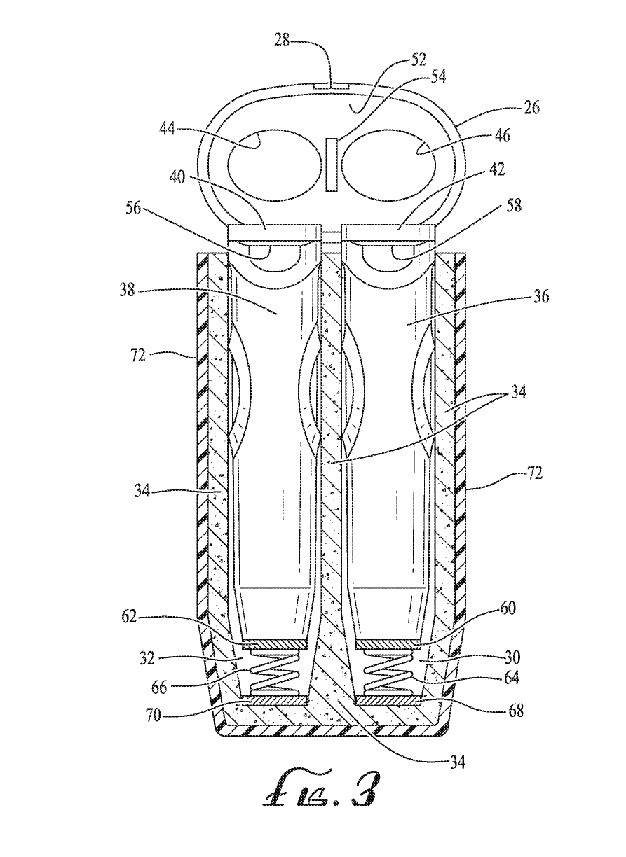

Interior containers, or alternatively, cavities 30, 32 are formed inside of the case 20. Insulation 34 surrounds the cavities 30, 32 as shown in FIG. 3 and functions to maintain the temperature of the epinephrine at room temperature for a reasonable time in elevated temperature environments. The insulation material is preferably foam insulation, such as closed cell polyurethane foam. Other materials may be used so long as it accomplishes the objective of preventing the epinephrine to be rendered useless due to elevated temperatures.

With reference to FIG. 3 two EpiPen® brand epinephrine auto-injectors 36, 38 are shown in side-by-side relationship. As shown in Figures and 3 each of the containers 36, 38 has a top cap 40, 42. The top caps 40, 42 fit within shallow cavities 44, 46 in the body of case top cap 26. Each of the caps 40, 42 for the individual containers also have rapid opening caps and cap tabs 56, 58. As shown in FIG. 2 the case top cap 26 has double, flexible hinges 48, 50 that join the top cap 26 to the back side of the body 22. The individual caps 40, 42 preferably have flexible hinges, of the same type as hinges 48, 50. Cavities 44, 46 are preferably formed in insulation 52, which in turn is positioned in top cap 26.

A conventional temperature measuring strip 54 is placed inside of the case, preferably on the underside of the top cap 26 and on the insulation 52. The temperature measuring strip functions to monitor the temperature inside of the case, which approximates the temperature of the encased epinephrine. Preferably the strip 54 is of the type that provides an indication of the highest temperature reached inside of the case, thus providing an indication on whether the epinephrine has been heated to a temperature high enough to render the epinephrine ineffective. In an alternate embodiment, single use strips are used so that a replacement strip is placed in the case upon replacement of the auto-injector.

Each individual container 36, 38 has a spring loaded bottom flange 60, 62. Springs 64, 66 are anchored to container bottom plates 68, 70. The springs 64, 66 function to cause the encased injector 36 or 38 to pop up immediately upon opening the cap 26 and facilitate grasping of either injector 36 or 38 by the user when in urgent need of an epinephrine injection.

The case 22 is preferably made of a hard material, of a predetermined thickness and its outer surface 72 (FIGS. 3 and 4) of a reflective material. In one alternate embodiment the back side of the case 20 has a belt clip. Other alternate embodiments could include loops for a shoulder strap, or other type of mounting or carrying structures such as clips, Velcro® brand fasteners.

The case housing is sized to include conventional insulation material outside of the auto-injector cavities and inside of the housing. Preferred insulation would be foam insulation, such as closed cell polyurethane foam. The volume of the space inside of the housing for the insulation in combination with the specific insulation capability of the chosen insulation material is sufficient to prevent over-heating of the contained epinephrine for sustained periods of high ambient temperatures, such as would be experienced inside of a glove compartment in an automobile during the summer in a warm climate. The amount or volume of insulation material preferably should be sufficient to maintain the encased auto-injector(s) at room temperature, that is about 65-75° F. during the daytime when the case is exposed to the sun, or in the glove compartment of a car or truck. The case 20 is made of, preferably, a hard, durable material such as ABS plastic to withstand dropping and other blunt force contacts.

Caps 40, 42 are of any conventional design, such as the type of pop-top or snap-open caps that are provided on conventional EpiPen® brand epinephrine auto-injectors.

During use an EpiPen® brand epinephrine auto-injector is placed in each of the cavities or internal containers 30, 32 and pressed downward to move the plates 60, 62 downward and compress the springs 64, 66 against the bottoms or plates 68, 70. External cap 26 is then closed over the two internal caps 40, 42. The injector-loaded case may then be carried by a user or stored in a convenient place such as a backpack, glove compartment, etc. When a need arises, such as the onset of an allergic reaction, the user can quickly open outer cap 26, and quickly retrieve one of the injectors. Upon opening top cap 26, the contained auto-injectors will pop out as the spring 64 or 66 uncoils and will present the injectors for easy grasping by the user. The user then opens the auto-injector cap and uses the auto-injector in accordance with it's and the user's doctor's instructions.

After loading and until use, the case protects the auto-injectors from physical damage and also protects the epinephrine from degradation due to heat or cold.

Conventional cases for EpiPen® brand epinephrine auto-injectors are known and one embodiment is shown in FIG. 5. Such cases are made of a hard plastic and are not insulated. Each case typically has a rapid opening (pop-top) cap.

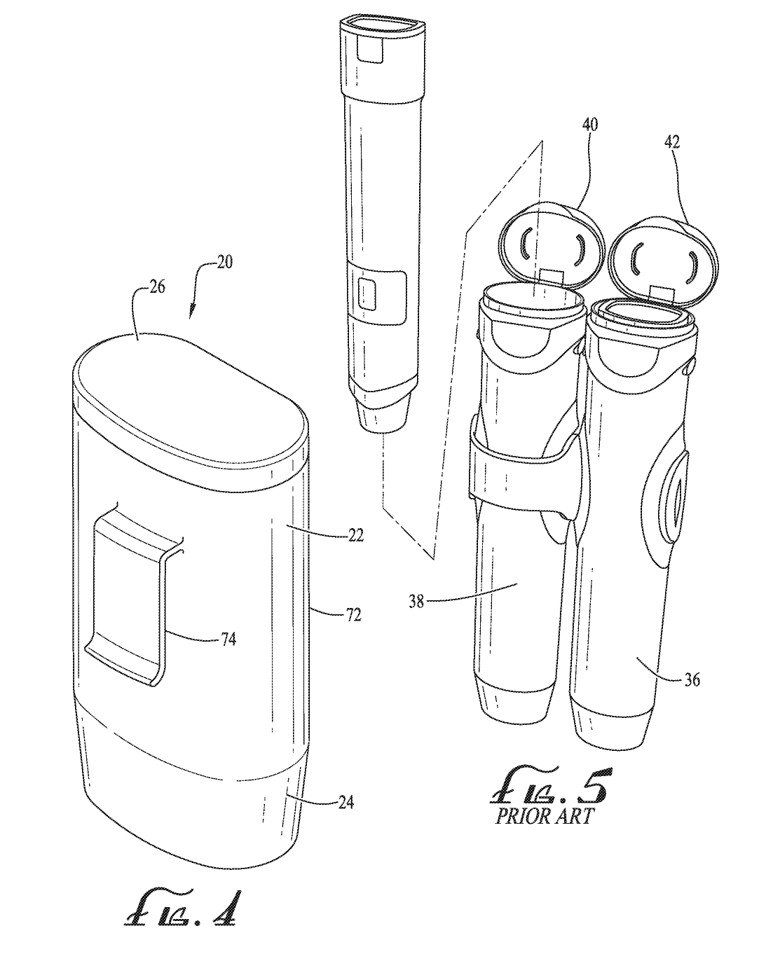

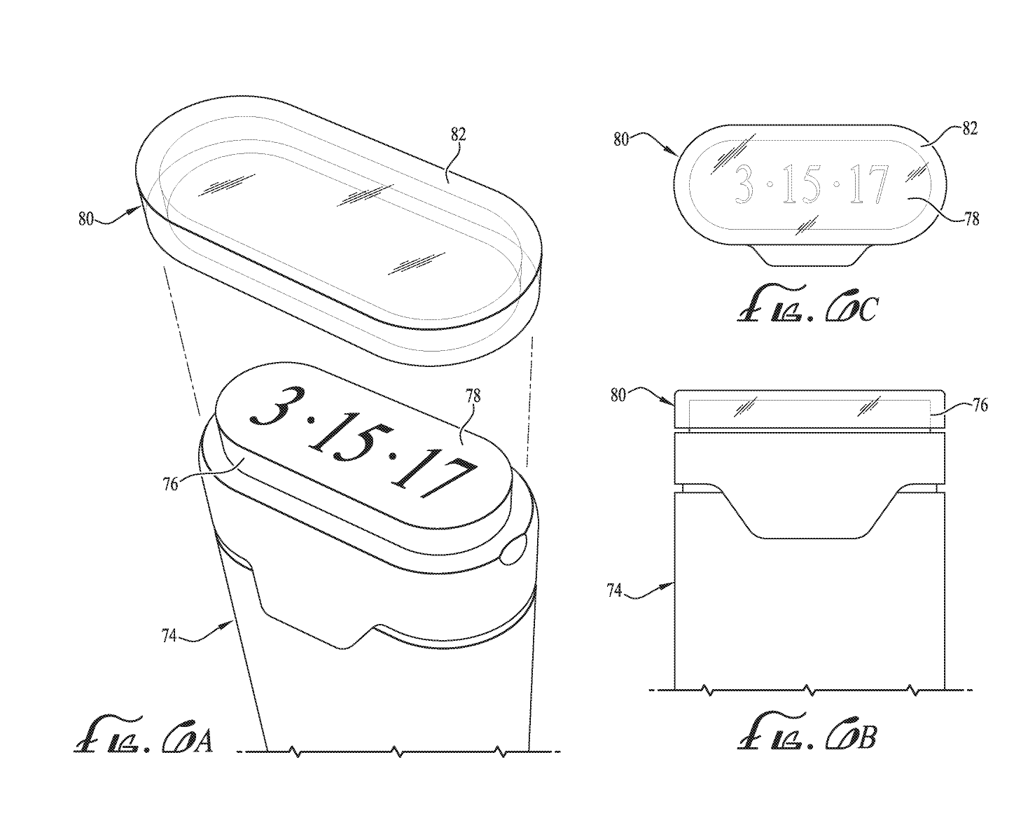

FIGS. 6A, 6B and 6C illustrate an alternate embodiment case 74 that includes a plate 76 on the top of the case. The top surface 78 is preferably made of a material upon which information can be written, erased and re-written. The surface 78 most preferably is of a conventional “white board” material. The re-writeable plate 76 is intended to be used to write the expiration date of the encased epinephrine injector(s) so that the user will have a readily available indication of when the encase auto-injector is near its expiration date, and should be replaced with a fresh auto-injector. FIG. 6A shows the case 74, the plate 76 and a nominal, expiration date of 3-15-17 written on top surface 78. A cap 80 covers the plate 76. The cap 80 preferably has a clear top surface 82 to enable the user to readily see the underlying expiration date. Preferably the plate 76 has a predetermined height, with its top surface 78 made of re-writeable material. Cap 80 is sized to snugly fit over the plate 76 and to protect the writing on the top surface from being rubbed off. Preferably the top 82 of the cap 78 is of a clear, conventional plastic material. FIG. 6B shows the plate 76 and the top 82 of cap 80 in side view, and FIG. 6C shows the nominal expiration date visible through the clear top 82 of the top cap 80.

As will be appreciated by those skilled in this field, other writable means can be used to serve as a reminder of the expiration date. For example, a paper or plastic tab could be used as the surface upon which the expiration date is written, and the case could then have a small frame fixed to the outer surface of the case, and into which the paper or plastic tab would be inserted. If a paper tab is used, then a new paper tab preferably would be used to mark the expiration date of each new replacement auto-injection. If a plastic tab is used, the old expiration date would simply be wiped off and a new date would be written on the tab with an erasable marker.

As will also be appreciated by those skilled in this art, the location of the tab or re-writable surface can vary. For example, the re-writable surface, tab, frame or plate could be positioned on the top, as shown, or on the front side, back side, bottom or side surfaces of the case. Alternatively, the re-writable surface could be placed on the inside of the case. The re-writable surface could be positioned on virtually any surface of the case, so long as the re-writable surface could function for its intended purpose of having an expiration date of the encased epinephrine recorded on the surface and available to the user.

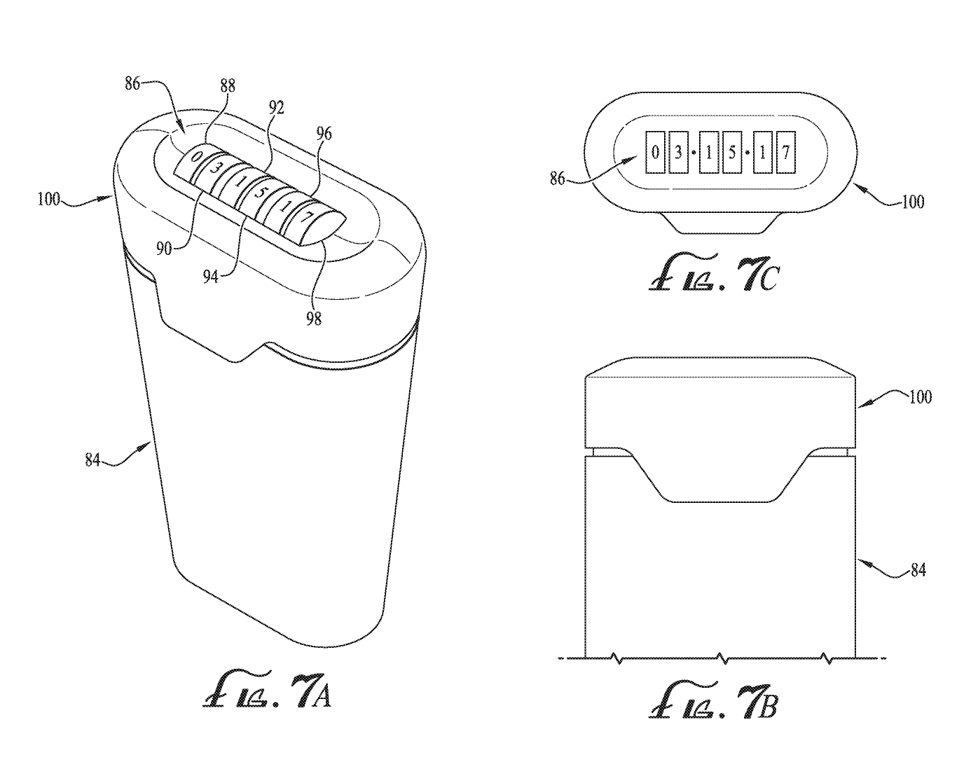

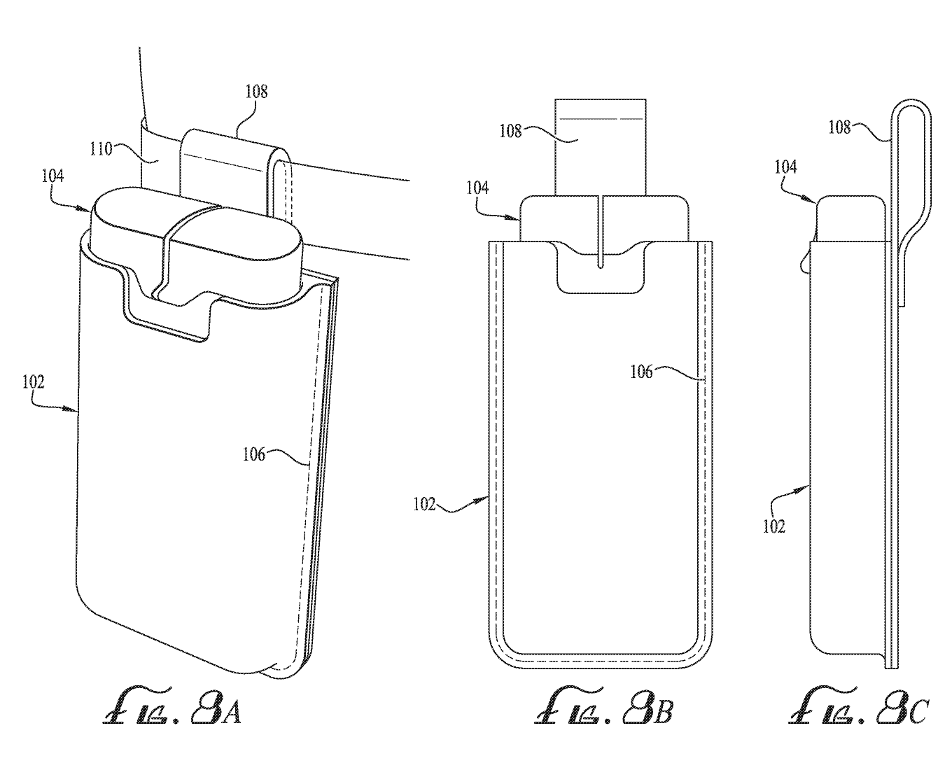

Referring to FIGS. 7A-7C an alternate, combination type case 84 is described. In this alternate embodiment case 84 preferably includes a 6-number combination set of wheels or dials positioned in a holder 86 on the case 84. As shown in FIGS. 7A-7C the holder 86 is positioned on top of the case and embedded in top cap 100, but, as will be appreciated by those skilled in this art, the holder 86 could be placed on the case at other locations, such as front surface, side surfaces, etc., as well as inside of the case. The holder and the dials are of a conventional design. In one embodiment, as shown, six dials 88, 90, 92, 94, 96 and 98 are shown. In the example shown, an expiration date of 03-15-17 is used. Alternatively, other means could be used to indicate an expiration date with use of wheels. For example, in one alternate embodiment, four wheels could be used, with one wheel having numbers 1 through 12 to indicate month, two wheels used to indicate a 2-digit day of the month, and a fourth wheel having several 2-digits to represent a number of years into the future, such as 17, 18, 19, 20, etc. to represent 2017, 2018, 2019, 2020, etc. As will also be appreciated, the orientation of the wheels could vary, with the axis of rotation of the wheels being at right angles to those shown for the FIGS. 7A-7C wheels. As shown the axis of rotation of each wheel or dial is perpendicular to the longitudinal centerline of the case 84; in the alternate embodiment referred to the axis of rotation of each wheel or dial would be parallel to the longitudinal centerline. During use, the user would simply dial in the expiration date of the auto-injector that is currently in use and in the case. Referring to FIGS. 8A-8C an alternate stitched holster with belt clip 102 is shown. Auto-injector 104 is shown placed in the holster 102, and stitches 106 are shown along the edges of holster where the holster material is stitched together. The holster material can be leather, a durable cloth, a durable polymeric material, or virtually any other material that will function as a holster. In other words, as long as the holster contains the auto-injector case and does not cause any adverse effect, virtually any material of construction may be used for the present invention. The holster 102 has belt loop or clip 108 positioned on the back side of the holster 102. The holster then may be attached to belt 110 during use, such as for hiking, walking, etc. As will be appreciated by those skilled in this art, the belt loop may be of any conventional design, may be unitary with the holster, or may be removable from the holster.

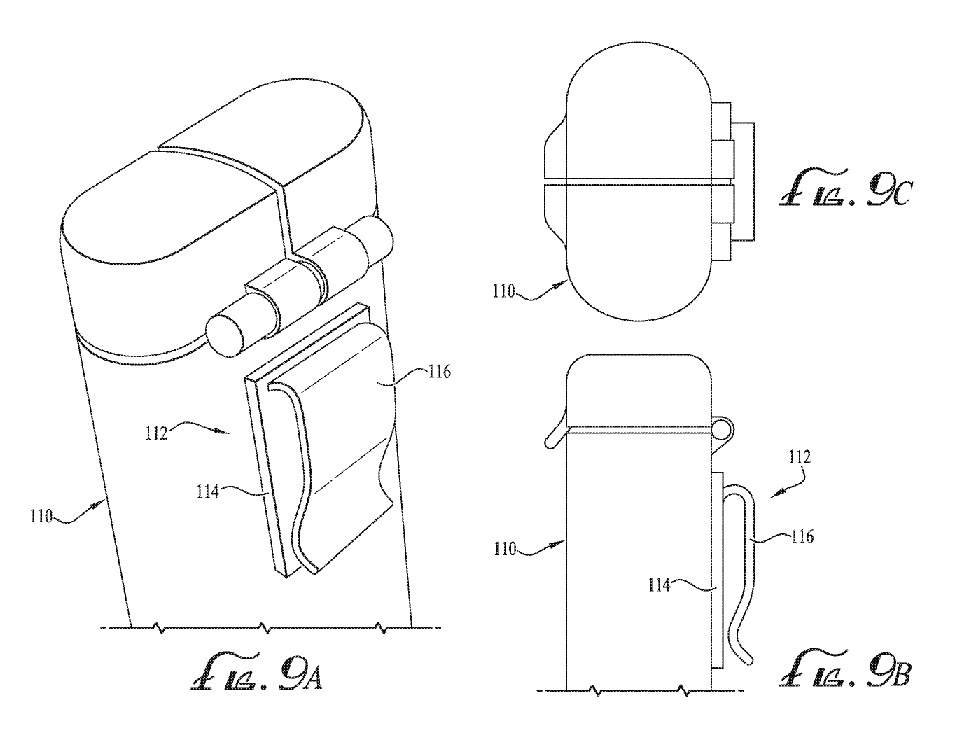

Referring to FIGS. 9A-9C another alternate embodiment removable/replaceable belt clip case 110 shown. Clip assembly 112 is shown attached to the back side of case 110. The clip assembly preferably includes a base plate 114 and a clip hook 116. As will be appreciated by those skilled in this art, the base plate 114 and clip hook 116 may be of a unitary construction, or may be made of separate components, such that the hook is removably retained by the base place by a conventional design, such as tongue and groove, snap lock, Velcro® brand fastener, etc. The clip hook 116 is preferably made of a strong material, and is preferably shaped with a curved top area that functions to provide strain relief as the clip is placed on and removed from a belt.

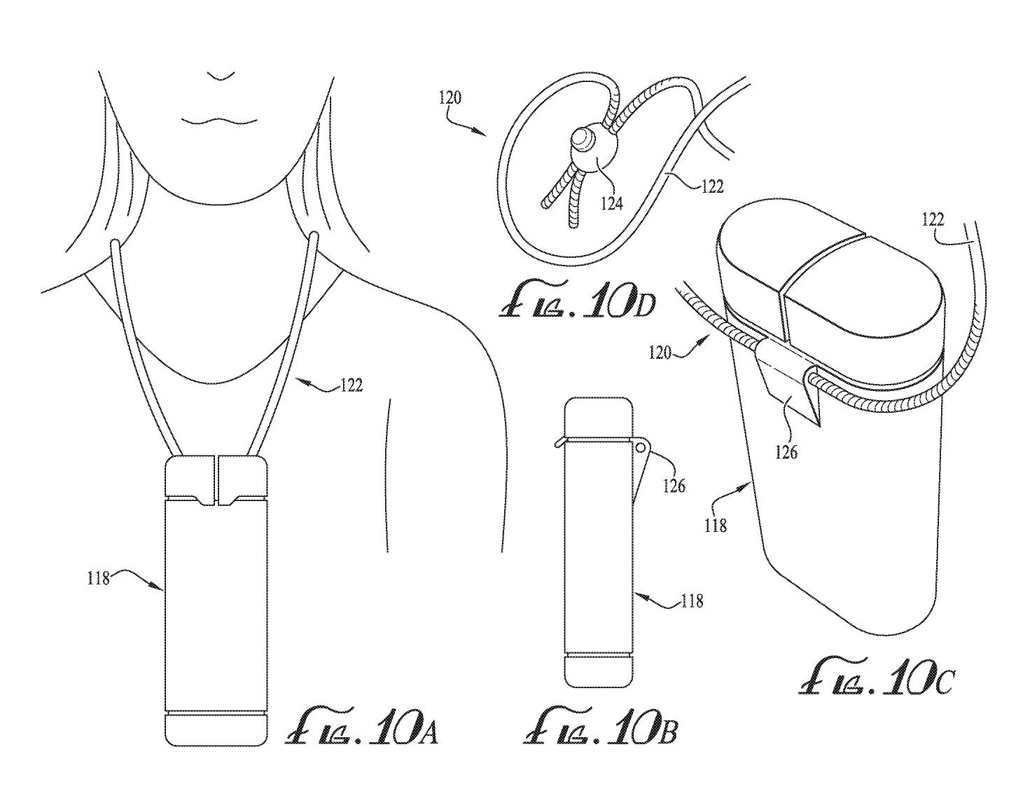

Referring to FIGS. 10A-10D another alternate embodiment, an adjustable lanyard case 118 is shown. The lanyard case 118 includes a conventional lanyard assembly 120 that includes conventional lanyard cord 122 and conventional lanyard fastener 124. The lanyard cord may be made of conventional material such as leather, nylon, rope, twine or other durable material that can be used to hold the case around the neck of a user, as shown in FIG. 10A. The lanyard fastener can be of virtually any design, such as a spring-loaded clip, so long as it functions to adjustably hold the case in position around a user's neck as shown in FIG. 10A. The lanyard cord 122 is threaded through loop 126 that is fixed to or part of the case 118.

Although specific embodiments of the invention have been described, various modifications, alterations, alternative constructions, and equivalents are also encompassed within the scope of the invention.

The specification and drawings are, accordingly, to be regarded in an illustrative rather than a restrictive sense. It will, however, be evident that additions, subtractions, deletions, and other modifications and changes may be made thereunto without departing from the broader spirit and scope of the invention as set forth in the claims.

What is claimed is:

1. A case for holding two epinephrine auto-injectors comprising:
a housing having a length, width, height, top end, top end cap, bottom end, and two cavities extending from said top end toward said bottom end;
insulation material positioned around said cavities, inside of said housing and adapted to maintain the interior of said case at ambient temperature for a predetermined period;
a temperature measuring strip positioned inside of said housing and adapted to measure and to provide an indication of the highest temperature reached inside of said device;
each of said cavities sized and configured to encase an epinephrine auto-injector;
each of said cavities having an internal top cap;
a spring-loaded ejector assembly positioned at the bottom of each of said cavities;
said ejector assembly including a movable plate, a spring and adapted to move toward the bottom of said housing upon insertion of an auto-injector and to move said auto-ejector toward the top end of said housing when a corresponding internal top cap; and,
said case including means for recording an expiration date of an epinephrine auto-injector contained within said case.

2. An insulated epinephrine auto-injector system comprising:
an epinephrine auto-injector container having a first container end and a longitudinal centerline extending from the first container end to a second container end, an epinephrine auto-injector and an auto-injector container top cap positioned adjacent the first container end;
said auto-injector container having sides of a predetermined length from the first container end to the second container end, a predetermined outer periphery, said auto-injector container top cap is connected to the auto-injector container at the first container end by a hinge and rotatable about an axis perpendicular to the container longitudinal centerline, a closed bottom at the second container end and a cavity open at the first container end;
said epinephrine auto-injector including an auto-injector top cap, a substantial part of said auto-injector positioned in said cavity;
thermal insulation positioned between said cavity and said container outer periphery, and surrounding said auto-injector and said auto-injector top cap; and,
and adjustable lanyard assembly including a lanyard cord, a lanyard fastener and a loop attached to said case, said lanyard assembly adapted to be adjustable to various loop sizes and to hold the container around a user's neck.

3. The auto-injector system of claim 2 further including a temperature measuring strip positioned in the case.

4. The case of claim 1 wherein said insulation is closed cell foam.
